(19) United States
(12) Patent Application Publication
(10) Pub. No.: US 2017/0036993 A1
(43) Pub. Date: Feb. 9, 2017

(54) METAL-ORGANIC FRAMEWORK FOR FLUID STREAM FILTRATION APPLICATIONS

(71) Applicant: Eastman Chemical Company, Kingsport, TN (US)

(72) Inventors: Meera Angayarkanni Sidheswaran, Jonesborough, TN (US); Guy Ralph Steinmetz, Kingsport, TN (US); Lori Cooke Ensor, Blountville, TN (US)

(73) Assignee: Eastman Chemical Company, Kingsport, TN (US)

(21) Appl. No.: 14/820,593

(22) Filed: Aug. 7, 2015

(51) Int. Cl. C07C 229/76; B01J 20/22; B01J 31/16; C07C 227/18; B01J 37/02; B01J 37/04; B01J 20/30; B01D 53/04; B01J 37/03

(52) U.S. Cl. CPC C07C 229/76; B01D 53/04; B01J 20/226; B01J 31/1691; B01J 37/031; B01J 37/0236; B01J 37/04; B01J 20/30; C07C 227/18; B01D 2257/70; B01D 2253/204; B01J 2531/26

(57) ABSTRACT

The present invention relates to a porous metal-organic framework (MOF) and includes a process for making the MOF and a process for using the MOF to remove aldehyde from a fluid stream. The MOF comprises a uniform and reproducible structure that can be synthesized at room temperature. The MOF is highly effective at removing an aldehyde from a fluid stream.

### FIELD OF THE INVENTION

The present invention relates to a porous metal-organic framework (MOF) and includes a process for making the MOF and a process for using the MOF to remove aldehyde from a fluid stream.

### BACKGROUND

Metal-organic frameworks are crystalline microporous materials that are useful in many industries. MOFs have strong bonding properties and provide a geometrically well-defined structure with high surface area and pore volume. MOFs may be produced by mixing a metal with an organic ligand. MOFs have wide-ranging applications and can be used as catalysts in organic reactions. MOFs may be used in separation materials, gas purification, filtration, ion-exchange, and processes involving removal of impurities from industrial aqueous streams, removal of impurities from hydrocarbon streams, removal of color from paper mill waste waters, removal of metals from aqueous solutions, removal of metals from hydrocarbon solutions, removal of hydrocarbon contaminants from aqueous systems, and removal of hydrocarbon contaminants from hydrocarbon systems.

Typically, MOF synthesis requires precipitating the MOF in solution over an extended period of time under high temperature conditions (hydrothermal or solvothermal synthesis). For example, in Amino-based metal-organic frameworks as stable, highly active basic catalysts (Jorge Gascon et al., 261 Journal of Catalysis 75 (2009)) (hereinafter “Gascon”) precipitation step required the solution be “heated in an oven at 373 K for 24 h, yielding cube-shaped crystals.” Other synthesis methods require heating the resulting sample over multiple days. Thus, there is a need in the art for an amino-based MOF that can be reproducibly synthesized under room temperature conditions.

As explained in A spray-drying strategy for synthesis of nanoscale metal-organic frameworks and their assembly into hollow superstructures (Arnau Carné-Sánchez et al., 5 Nature Chemistry 203 (2013)), spherical particles have a benefit over cubic or rhombic structures because spherical particles enable “simultaneous encapsulation of active species in the cavities of the MOF” and provide a more stable MOF. Thus, there is a need in the art for an amino-based MOF that comprises spherical particle structure.

The present invention addresses this need as well as others that will be apparent from the following description and claims.

### SUMMARY

In a first embodiment, the present invention provides a MOF prepared by a process comprising the steps of (1) mixing an organic ligand with a metal ion in a first solvent to form a first solution, (2) adding an amine to the first solution to precipitate the MOF and form a first suspension, (3) separating the MOF from the first suspension, and (4) drying the MOF. In one aspect, the MOF is produced at room temperature conditions. In one aspect, the MOF comprises essentially spherical particles having a porous structure.

In a second embodiment, the present invention provides a method for synthesizing a MOF comprising the steps of (1) mixing an organic ligand with a metal ion in a first solvent to form a first solution, (2) adding an amine to the first solution to precipitate the MOF and form a first suspension, (3) separating the MOF from the first suspension, and (4) drying the MOF. In one aspect, the separating step comprises filtering and washing the MOF, and the separating step may be repeated more than one time.

In a third embodiment, the present invention provides a method for removing an aldehyde from a fluid stream by providing a MOF and contacting the MOF with the fluid stream. The MOF is prepared by a process comprising the steps of (1) mixing aminoterephthalic acid with a zinc nitrate solution in a first solvent to form a first solution, (2) adding triethylamine to the first solution to precipitate the MOF and form a first suspension, (3) separating the MOF from the first suspension, and (4) drying the MOF.

### BRIEF DESCRIPTION OF THE DRAWINGS

FIG. 1 is the scanning electron microscopy image of the ZnA-MOF of Example 1, magnified 2,500 times.

FIG. 2 is a cross-sectional view of the ZnA-MOF of Example 1, magnified 100,000 times.

### DETAILED DESCRIPTION

It has been discovered that a metal-organic framework (MOF) can be produced by a process using room temperature precipitation rather than high temperature processes used previously. In addition, it has been discovered that the resulting MOFs have a substantially uniform and reproducible structure. Further these MOFs can be used to filter trace components out of a fluid stream to a high level of efficiency.

In a first embodiment, the present invention provides a MOF prepared by a process comprising the steps of (1) mixing an organic ligand with a metal ion in a first solvent to form a first solution, (2) adding an amine to the first solution to precipitate the MOF and form a first suspension, (3) separating the MOF from the first suspension, and (4) drying the MOF. In one aspect, the MOF is produced at room temperature conditions. In one aspect, the MOF comprises essentially spherical particles having a porous structure.

It is to be understood that the mention of one or more process steps does not preclude the presence of additional process steps before or after the combined recited steps or intervening process steps between those steps expressly identified. Moreover, the lettering or numbering of process steps or ingredients is a convenient means for identifying discrete activities or ingredients and the recited lettering or numbering can be arranged in any sequence, unless otherwise indicated.

The first step of producing the MOF comprises mixing an organic ligand with a metal ion in a first solvent to form a first solution. The organic ligand can be a monodentate or polydentate organic ligand. In one aspect, the organic ligand is bidentate, tridentate, or tetradentate. Non-limiting examples of the organic ligand include aminoterephthalic acid, terephthalic acid, 1,2,3-benzenetricarboxylic acid, 1,3,5-benzenetricarboxylic acid, or 2,2′-bipyridine-5,5′-dicarboxylic acid. In one aspect, the organic ligand comprises aminoterephthalic acid.

The metal ion can be in the form of a metal salt or aqueous solution. Non-limiting examples of the metal ions include zinc, copper, cerium, nickel, manganese, platinum, or iron. In one aspect the metal ion is zinc.

The first solvent can be used to dissolve the metal ion and the organic ligand to form a first solution. Non-limiting examples of the first solvent include dimethylformamide, diethylformamide, or dibenzylformamide. In one aspect, the first solvent comprises dimethylformamide.

The second step of producing the MOF comprises adding an amine to the first solution to precipitate the MOF and form a first suspension. Non-limiting examples of the amine include methylamine, ethylamine, n-propylamine, iso-propylamine, n-butylamine, sec-butylamine, iso-butylamine, tert-butylamine, n-pentylamine, neo-pentylamine, n-hexylamine, pyrrolidine, cyclohexylamine, morpholine, pyridine, 8-azaphenanthrene, 1,4-diaminobenzene, or triethylamine. In one aspect, the amine is selected from the group consisting of methylamine, ethylamine, n-propylamine, iso-propylamine, n-butylamine, sec-butylamine, iso-butylamine, tert-butylamine, and triethylamine. In one aspect the amine comprises triethylamine. In one aspect, the amine is added at room temperature conditions.

The precipitate will form as the amine is added to the first solution, forming a first suspension. In one aspect the first suspension comprises the precipitate and the remainder of the first solution after precipitation. In one aspect, the precipitate is a pale-yellow solid. The first suspension may be stirred or left unstirred for a period of time. In one aspect, the first suspension is stirred continuously for up to 2 hours, up to 4 hours, or up to 8 hours. In one aspect, the first suspension can be left at room temperature for up to 12 hours, up to 24 hours, or up to 48 hours between the precipitating step and the separating step.

The third step of producing the MOF comprises separating the MOF from the first suspension. One of ordinary skill in the art recognizes the need to separate the newly precipitated MOF from the first suspension. In one aspect, the separating step comprises a first filtering of the MOF out of the first suspension, a first washing of the MOF with a second solvent, and a second filtering of the MOF. In one aspect, the first washing and second filtering of the separating step are completed separately by adding the solvent and stirring the newly made suspension followed by filtering. In one aspect the first washing and second filtering of the separating step are completed simultaneously by pouring the solvent over the MOF on a filter. The separating step may also further comprise a second washing of the MOF with a third solvent and a third filtering of the MOF. In one aspect, the separating step is repeated at least one time. In one aspect, the separating step is repeated at least twice. In one aspect, the second washing and the third filtering are repeated at least one time.

In one aspect, the second solvent is used to wash the resulting MOF. Non-limiting examples of the second solvent include ethanol, dimethylformamide, dichloromethane, toluene, methanol, chlorobenzene, diethylformamide, methylamine, acetonitrile, benzyl chloride, or ethylene glycol. In one aspect, the second solvent is selected from the group consisting of ethanol, dimethylformamide, dichloromethane, methanol, and diethylformamide. In one aspect, the second solvent comprises dimethylformamide.

The first and second solvent can be chosen independently of each other. In one aspect the second solvent and the third solvent are the same composition.

In one aspect, the third solvent is used to wash the resulting MOF after the second filtering. In one aspect, the third solvent is used to wash the resulting MOF after the third filtering. Non-limiting examples of the third solvent include ethanol, dimethylformamide, dichloromethane, toluene, methanol, chlorobenzene, diethylformamide, methylamine, acetonitrile, benzyl chloride, or ethylene glycol. In one aspect, the third solvent is selected from the group consisting of ethanol, dimethylformamide, dichloromethane, methanol, and diethylformamide. In one aspect, the third solvent comprises dichloromethane.

The fourth step of producing the MOF comprises drying the MOF. The drying of the MOF may occur at a temperature ranging from room temperature to about 100° C. In one aspect, the drying step occurs at a temperature ranging from about 60° to about 70° C. The method of drying can vary and may include air-drying, vacuum drying, or other drying techniques known to one skilled in the art. In one aspect of the invention, the drying step comprises vacuum drying at a temperature of 60° to 70° C.

The resulting MOF may be colorless or colored. In one aspect, the MOF crystals are pale yellow. The resulting MOF comprises particles that are essentially spherical in shape. The term “essentially spherical” as used herein means that the material has a morphology that includes spherical, as well as oblong, and the like and can have surface irregularities. In one aspect of the invention at least 90% of the essentially spherical particles have a diameter ranging from 10 μm to 20 μm. In one aspect, the particles have a diameter ranging from 14 μm to 17 μm.

In a second embodiment, the present invention provides a method for synthesizing a MOF comprising the steps of (1) mixing an organic ligand with a metal ion in a first solvent to form a first solution, (2) adding an amine to the first solution to precipitate the MOF and form a first suspension, (3) separating the MOF from the first suspension, and (4) drying the MOF. In one aspect, the separating step comprises filtering and washing the MOF, and the separating step may be repeated more than one time.

The description of the MOF and process for making the MOF herein above, such as, for example, the description of the mixing step, adding an amine step, the separating step, the drying step, the metal ion, the organic ligand, the first solvent, the amine, the first filtering, the first washing, the second solvent, the second filtering, the second washing, the third solvent, the third filtering, and the essentially spherical particles, also apply to the process for synthesizing the MOF.

For example, in one aspect, non-limiting examples of the organic ligand include aminoterephthalic acid, terephthalic acid, 1,2,3-benzenetricarboxylic acid, 1,3,5-benzenetricarboxylic acid, and 2,2′-bipyridine-5,5′-dicarboxylic acid; non-limiting examples of the metal ion include zinc, copper, cerium, nickel, manganese, platinum, and iron; and non-limiting examples of the amine include methylamine, ethylamine, n-propylamine, iso-propylamine, n-butylamine, sec-butylamine, iso-butylamine, tert-butylamine, n-pentylamine, neo-pentylamine, n-hexylamine, pyrrolidine, cyclohexylamine, morpholine, pyridine, 8-azaphenanthrene, and triethylamine. In one aspect the organic ligand is aminoterephthalic acid, the metal ion is zinc, and the amine is triethylamine. In one aspect, the amine is added at room temperature.

In one aspect, the separating step comprises (a) a first filtering of the MOF out of the first suspension, (b) a first washing of the MOF with a second solvent, and (c) a second filtering of the MOF. In one aspect, non-limiting examples of the first solvent include dimethylformamide, diethylformamide, and dibenzylformamide; and non-limiting examples of the second solvent include ethanol, dimethylformamide, dichloromethane, toluene, methanol, chlorobenzene, diethylformamide, methylamine, acetonitrile, benzyl chloride, and ethylene glycol.

In one aspect, the organic ligand comprises aminoterephthalic acid, the metal ion comprises zinc, and the amine comprises triethylamine.

In one aspect, the MOF is in the form of essentially spherical particles. In one aspect, 90% of the particles have a diameter ranging from 10 μm to 20 μm.

In a third embodiment, the present invention provides a method for removing an aldehyde from a fluid stream by providing a MOF and contacting the MOF with the fluid stream. The MOF is prepared by a process comprising the steps of (1) mixing aminoterephthalic acid with a zinc nitrate solution in a first solvent to form a first solution, (2) adding triethylamine to the first solution to precipitate the MOF and form a first suspension, (3) separating the MOF from the first suspension, and (4) drying the MOF.

The description of the MOF and process for making the MOF herein above, such as, for example, the description of the mixing step, adding an amine step, the separating step, the drying step, the metal ion, the organic ligand, the first solvent, the amine, the first filtering, the first washing, the second solvent, the second filtering, the second washing, the third solvent, the third filtering, and the essentially spherical particles, also applies to the method of removing aldehyde from a fluid stream.

In one aspect, the triethylamine is added at room temperature.

In one aspect, the separating step comprises (a) a first filtering of the MOF out of the first suspension, (b) a first washing of the MOF with a second solvent, and (c) a second filtering of the MOF. In one aspect, the first solvent comprises dimethylformamide, diethylformamide, or dibenzylformamide. In one aspect, the second solvent comprises ethanol, dimethylformamide, dichloromethane, toluene, methanol, chlorobenzene, diethylformamide, methylamine, acetonitrile, benzyl chloride, or ethylene glycol.

In one aspect, the MOF is in the form of essentially spherical particles. In one aspect, 90% of the particles have a diameter ranging from 10 μm to 20 μm.

In one aspect, the fluid stream comprises a gas stream. Non-limiting examples of the fluid stream include air, water, tobacco smoke, or cigarette smoke.

In one aspect the MOF contacts the fluid stream and the MOF chemically or physically adsorbs, absorbs, entraps, catalyzes, or chemically reacts with the aldehyde in the fluid stream. In one aspect, the contacting the fluid stream comprises forcing the fluid stream through a material which includes the MOF. In one aspect, the material which includes the MOF comprises cellulose acetate.

The aldehyde may be a single aldehyde or a mixture of various aldehydes. Non-limiting examples of the aldehyde include acetaldehyde, crotonaldehyde, formaldehyde, acrolein, butyraldehyde, benzyl aldehyde, propionaldehyde, or combinations thereof. In one aspect, the aldehyde comprises acetaldehyde, crotonaldehyde, formaldehyde, or a combination thereof.

In one aspect of the invention, the aldehyde comprises acetaldehyde. Using the MOF according to the present invention, at least 50% of the acetaldehyde is removed from the fluid stream. In one aspect, at least 90% of the acetaldehyde is removed from the fluid stream. In one aspect, 99% of the acetaldehyde is removed from the fluid stream. In one aspect, the MOF is capable of removing acetaldehyde in the range of 13,000 to 24,000 micrograms of acetaldehyde per gram of MOF. In another aspect, the MOF is capable of removing acetaldehyde in the range of 16,000 to 21,000 micrograms of acetaldehyde per gram of MOF.

In one aspect of the invention, the aldehyde comprises crotonaldehyde. Using the MOF according to the present invention, at least 50% of the crotonaldehyde is removed from the fluid stream. In one aspect, at least 90% of the crotonaldehyde is removed from the fluid stream. In one aspect, 99% of the crotonaldehyde is removed from the fluid stream. In one aspect, the MOF is capable of removing crotonaldehyde in the range of 1200 to 3300 micrograms of crotonaldehyde per gram of MOF. In another aspect, the MOF is capable of removing crotonaldehyde in the range of 1800 to 2500 micrograms of crotonaldehyde per gram of MOF.

In one aspect of the invention, the aldehyde comprises formaldehyde. Using the MOF according to the present invention, at least 50% of the formaldehyde is removed from the fluid stream. In one aspect, at least 90% of the formaldehyde is removed from the fluid stream. In one aspect, 99% of the formaldehyde is removed from the fluid stream. In one aspect, the MOF is capable of removing formaldehyde in the range of 18,000 to 69,000 micrograms of formaldehyde per gram of MOF. In another aspect, the MOF is capable of removing formaldehyde in the range of 30,000 to 50,000 micrograms of formaldehyde per gram of MOF.

In another aspect of the invention, the present invention provides for a process for embedding the MOF in cellulose acetate fibers. Embedding the MOF in cellulose acetate fibers forms a chemical bond between the MOF and the cellulose acetate. The MOF is embedded in the cellulose acetate fiber by a process comprising: (1) preparing the cellulose acetate fibers; (2) mixing the cellulose acetate fibers with a first solution comprising a metal ion; (3) adding an amine to the first solution; (4) separating the cellulose fibers embedded with the MOF; and (5) drying the MOF.

The description of the MOF and process for making the MOF herein above, such as, for example, the description of the mixing step, adding an amine step, the separating step, the drying step, the metal ion, the organic ligand, the first solvent, the amine, the first filtering, the first washing, the second solvent, the second filtering, the second washing, the third solvent, the third filtering, and the essentially spherical particles, also applies to the method of embedding the MOF in cellulose acetate fibers.

The preparation step comprises soaking cellulose acetate fibers in a solution comprising an acid and a base. The mixing step comprises soaking the cellulose acetate fibers in a solution comprising the metal ion. The mixing step further comprises adding an amine to the solution to precipitate the MOF and form a chemical attachment between the MOF and the cellulose acetate fibers. The separating step comprises using a solvent to filter and wash the cellulose fibers embedded with MOF. The separating step can be repeated multiple times as needed.

### EXAMPLES

These examples illustrate synthesis procedures for various absorbents and catalysts and testing procedures used to evaluate the effectiveness of both synthesized and commercial absorbents and catalysts. More specifically, the MOF synthesis procedures detail the process for synthesizing an amino-based MOF using a well-known technique and using the method as in the present invention. Further, the MOF effectiveness details a process of testing the aldehyde removal efficiency of the amino-based MOF of the present invention as well as various synthesized and commercial absorbents and catalysts. Lastly, the examples illustrate a process of embedding the MOF as in the present invention in cellulose acetate fibers by forming a chemical attachment.

Zinc nitrate, aminoterephthalic acid, dimethylformamide, anhydrous CH₂Cl₂, triethylamine, dichloromethane, methanol, NaMnO4.H₂O, MnSO₄, sodium permanganate, and Amberlyst® 36 were purchased from Sigma Aldrich. Cellulose acetate samples were Eastman Estron™ from Eastman Chemical Company. Silica samples were purchased from Aerosil®. Theta-alumina were purchased from Johnson Matthey. Zeolite Y CBV-600 and Zeolite Y CBV-901 were purchased from Zeolyst International. Calgon Carbon powder (CAS #7440-44-0, type: PCB-P) was purchased from Calgon Carbon Corporation. All materials were used as received from the vendors.

### Absorbent and Catalyst Synthesis

### Comparative Example 1

### Synthesis of a MOF Using Gascon Method

This example illustrates the synthesis of an amino-based MOF using the method described in Gascon (Jorge Gascon, Amino-based metal-organic frameworks as stable, highly active basic catalysts, 261 JOURNAL OF CATALYSIS 75 (2009)). First 15 mmol zinc nitrate hexahydrate and 5 mmol 2-aminoterephthalic acid were dissolved in 490 mL of dimethylformamide (“DMF”) and 10 mL water in a 600 mL Erlenmeyer flask equipped with a pressure-releasing device. The reaction mixture was heated in an oven at 373 K for 24 hours, precipitating cube-shaped crystals. The reaction vessel was then removed from the oven, allowed to cool to room temperature, and transferred to a nitrogen-filled glove box. The solvent was decanted and the remaining solid was washed six times with 50 mL of anhydrous DMF, each time letting the solid soak in the DMF for 8 hours. Then the solid was washed six times with 50 mL of anhydrous CH₂Cl₂, each time letting the solid soak in the CH₂Cl₂ for 8 hours. After the final CH₂Cl₂ wash, the solvent was decanted and the solid was placed under reduced pressure for 12 hours to remove the remaining CH₂Cl₂. This yielded pale-yellow cube-shaped crystals. Though the method itself was repeated, the crystal shape and size was not reproducible with each repetition. The BET surface area of the sample was measured as 7.6 m²/g.

### Example 1

### Synthesis of a MOF Using Room Temperature Acid-Base Method

This example illustrates the synthesis of a zinc-amino based MOF (ZnA-MOF) using room-temperature precipitation. A solution of 2 g of aminoterephthalic acid in 50 mL dimethylformamide (“DMF”) was added drop wise under constant stirring to a solution of 8 g zinc nitrate dissolved in 60 mL of DMF. To the resulting solution, 5 mL of triethylamine was added drop wise to precipitate the complex ZnA-MOF containing zinc oxide and active amine groups on the surface. The resulting precipitate, a pale yellow solid, was stirred continuously for 2 hours then left in the supernatant overnight at room temperature.

After 24 hours, the precipitate was filtered and washed with excess DMF. Then the precipitate was transferred into a clean beaker containing 50 mL dichloromethane (“DCM”). The precipitate was stirred in DCM for 2 hours then left in the DCM for 48 hours. These filter and wash steps were repeated two more times. After the filter and wash steps, the filtered ZnA-MOF was then vacuum dried at 60° to 70° C. overnight and stored in an airtight container in a low moisture environment, in which the moisture content was maintained at or below 20%.

The resulting ZnA-MOF was characterized using scanning electron microscopy (“SEM”), X-ray diffraction (“XRD”), energy dispersive spectroscopy (“EDS”), and Brunauer-Emmett-Teller (“BET”) surface area techniques (ASAP 2020, Micromeritics). The ZnA-MOF powder samples were fixed to a conductive carbon sticky pad on an aluminum sample stub for SEM-EDS analysis. The samples were imaged (uncoated) in an FEI Quanta 450F scanning electron microscope operating at low beam voltage (3-5 keV) and imaged using both the secondary electron Everhart-Thornley detector and the back scattered electron BSED detector. Elemental analysis was carried out using the Ametek EDAX Apollo XL 30 mm² detector attached to the FEI Quanta 450F scanning electron microscope operating at a beam voltage of 10 keV to collect energy dispersive spectra of the samples.

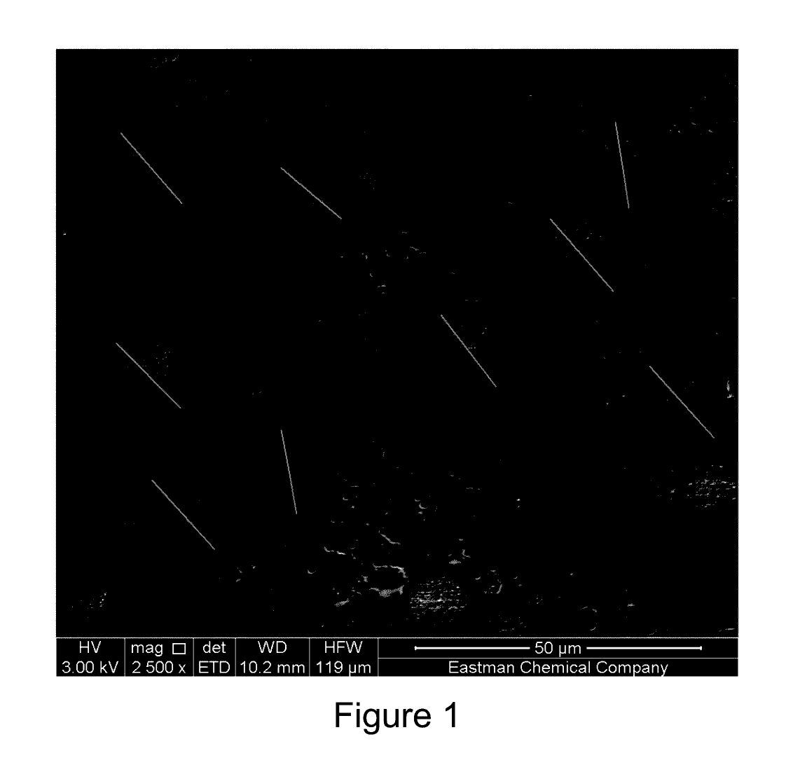

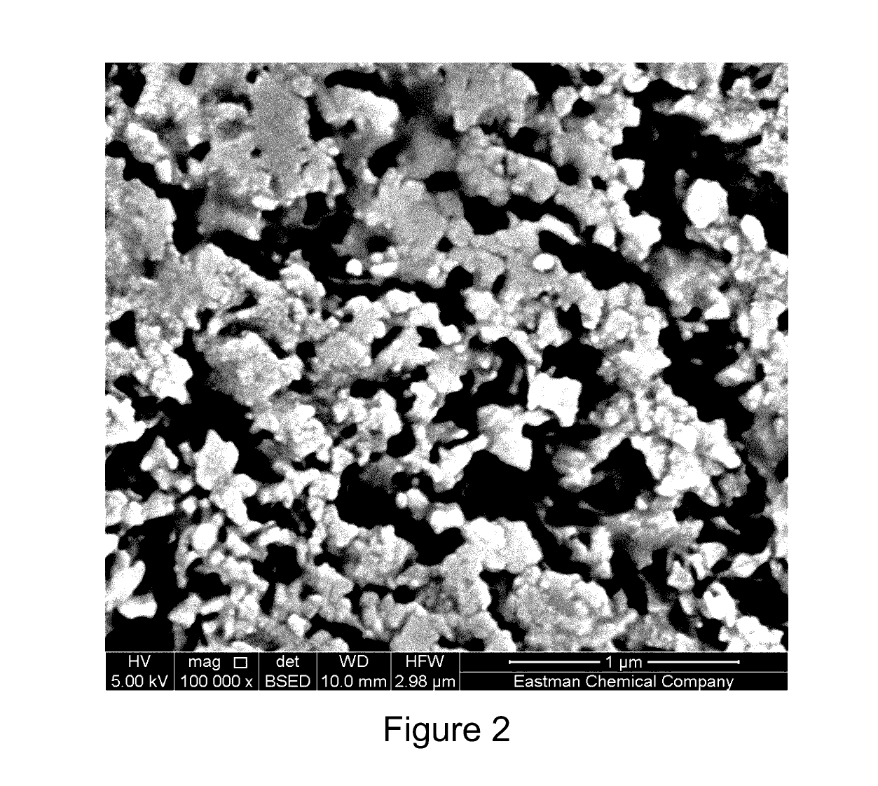

FIG. 1 shows an image obtained via scanning electron microscopy of the ZnA-MOF magnified 2500 times, showing a uniform ZnA-MOF particle size in the range of about 14 μm to about 17 μm. The needle structures in the image indicate that some zinc oxide precipitated out, which was confirmed by EDS results of the needle structures in the ZnA-MOF of Example 1. FIG. 2 shows a cross sectional view of the ZnA-MOF magnified 100,000 times, providing a visual image of the very porous structure.

The composition of the ZnA-MOF of Example 1 was confirmed by EDS analysis. EDS results of the needle structures show a peak for oxygen around 0.50 keV and a peak for zinc around 1.0 keV, confirming the needle structures are zinc oxide precipitate. EDS results of the surface of the ZnA-MOF spherical particle show a combination of zinc, oxygen, nitrogen, and carbon, confirming the composition of the ZnA-MOF. EDS results of a cross-section of the ZnA-MOF spherical particle sputtered with gold also show a combination of zinc, oxygen, nitrogen, carbon, and gold. Compared to the EDS of the surface of the ZnA-MOF, the EDS of the cross-section shows a larger carbon peak, similar nitrogen peak, and smaller zinc and oxygen peaks. These results show some variation in composition within the ZnA-MOF particles of Example 1.

The BET surface area of the ZnA-MOF produced using the Example 1 method was measured as 24.9 m²/g. This surface area measurement is low compared to the expected surface area of a typical MOF. The BET surface area is also lower than expected based on the images of the ZnA-MOF in FIG. 2 that shows a very porous structure. One skilled in the art recognizes that this low BET measured surface area is likely attributed to the inherent difficulty in obtaining surface area measurements for MOFs, as described in Nelson (Andrew P. Nelson et al., Supercritical Processing as a Route to High Internal Surface Areas and Permanent Micro porosity in Metal-Organic Framework Materials, 131 J. AM. CHEM. SOC. 458 (2009)). Further, the more accurate method for measuring MOF surface area employed in Nelson was not readily available and therefore not applied to the ZnA-MOF of Example 1.

As described in Nelson, the surface area was measured for four MOF materials. In Nelson, it was found that experimental BET surface areas frequently are less than theoretical surface areas, and these measurements often vary widely from one laboratory to another. Using BET surface area techniques, the measured surface areas for the four MOFs ranged from 36 to 1800 m²/g. Using the technique as described in Nelson, surface area measurements for those same samples increased to a range of 430 to 2850 m²/g, with each sample showing an increase ranging from 58% to as high as 1094%.

### Examples 2-3

### Repeated Synthesis of a MOF Using Room Temperature Acid-Base Method

The synthesis procedure of Example 1 was repeated two more times, each time producing a precipitate with consistent crystal structure. Each repetition of the Example 1 method produced a ZnA-MOF precipitate, with some excess zinc oxide precipitated out. The resulting ZnA-MOFs of Examples 2 and 3 had a particle size in the range of about 14 μm to about 17 μm, a fibrous outer shell, and a very porous structure, consistent with the findings of Example 1.

### Example 4

### Process of Embedding a MOF in Cellulose Acetate Fibers

This example illustrates the process of embedding the MOF to cellulose acetate fibers, creating a chemical attachment between the MOF and the cellulose acetate. 1 g cellulose acetate fibers (Eastman Estron™ acetate tow) were soaked in 1 M sodium chloroacetate and 5% sodium hydroxide for 1 hour. After 1 hour, the cellulose acetate fibers were washed three times with water then allowed to dry overnight at 40° C. The cellulose acetate fibers were then added to a solution of 1.6 g zinc nitrate in 4 mL dimethylformamide (“DMF”), 4 mL ethanol, and 4 mL water. The cellulose acetate fibers soaked in this solution overnight under stirring. Then a solution of 0.4 g aminoterephthalic acid in 10 mL DMF was added drop wise to the cellulose acetate-zinc nitrate solution and stirred vigorously for 2 hours. 0.5 mL triethylamine was added drop wise under stirring. Then the fibers soaked overnight in the mother liquor. After soaking overnight, the fibers were washed with 30 mL methanol three times and then dried at 80° C. overnight.

### Comparative Example 2

### Synthesis of a First Manganese Oxide Based Catalyst

This example illustrates the synthesis of a manganese oxide based catalyst used to filter aldehydes from air streams. A solution containing 18.9 g NaMnO₄.H₂O and 44.2 g of distilled deionized water was added drop wise to another solution containing 30.0 g MnSO₄ and 170 g of DD water in a 500 cc glass beaker at room temperature under agitation with a magnetic stirrer. The resulting slurry solution was stirred for 30 minutes then filtered to obtain the solids. The resulting solids were dried in a convection oven at 60° C. for 4 days.

The resulting manganese oxide based catalyst was characterized using scanning electron microscopy (“SEM”), tunneling electron miscroscopy (“TEM”), energy dispersive spectroscopy (“EDS”), and Brunauer-Emmett-Teller (“BET”) surface area techniques. The catalyst was initially degassed at 100° C. in nitrogen under vacuum, and the surface area analysis was performed using nitrogen under 77K. The BET surface area was estimated as 300.7 m²/g.

### Comparative Example 3

### Synthesis of a Second Manganese Oxide Based Catalyst

This example illustrates the synthesis of a manganese oxide based catalyst used to filter aldehydes from air streams. The procedure in Comparative Example 2 was followed, but after the resulting solid was dried at 60° C. for 4 days, the sample was then heated in an oven at 100° C. overnight. The sample was analyzed using the same procedure as in Comparative Example 2. The surface area was estimated as 260 m²/g.

### Comparative Example 4

### Synthesis of a Sodium Permanganate and Silica Based Catalyst

This example illustrates the synthesis of NaMnO₄—SiO₂-90 used to filter aldehydes from air streams. Two parts of 20% by weight solution of chemisorbent sodium permanganate in water was added to one part of silica Aerosil® and agitated for 3 hours. The excess solution was decanted and the resulting catalyst was dried at 60° to 100° C. for a period of time until the weight loss on the substrate was less than 10%.

### Comparative Example 5

### Synthesis of a Sodium Permanganate and Alumina Based Catalyst

This example illustrates the synthesis of NaMnO₄—Al₂O₃ used to filter aldehydes from air streams. The procedure in Comparative Example 4 was followed, but theta-alumina was used instead of silica Aerosil®.

### Absorbent and Catalyst Effectiveness

### Example 5

### Removal Efficiency of the MOF for Removing Acetaldehyde

A sample holder was developed to measure the removal efficiency of the ZnA-MOF and other absorbents and catalysts for removing acetaldehyde, crotonaldehyde, and formaldehyde. The sample holder was a 10-inch long, 0.25-inch inner diameter glass tube with 0.25-inch Swagelok fittings. A 2-cm long test bed was created by sandwiching approximately 0.2 g of the MOF between cellulose acetate fibers and housing the test bed in the sample holder. The downstream concentration of the various aldehydes was measured using a dinitrophenylhydrazine (“DNPH”) cartridge (Waters WAT047204) attached downstream of the sample holder. Air was pulled through the sample holder at a rate of 650 sccm using a peristaltic pump (Cole Parmer, Masterflex L/S precision drive 600 rpm). This yielded a face velocity of 0.35 m/s through the MOF bed. Before testing each sample, to ensure the system was running at steady state, blank measurements of the aldehyde to be tested were taken using a sample holder with only a cellulose acetate fiber test bed. After a blank measurement was obtained, the sample holder was switched out with a sample holder containing the MOF and cellulose acetate. Then air was pulled through the DNPH cartridges as described above for 15 to 20 minutes. The resulting concentration of the acetaldehyde in the outlet stream was determined using U.S. Environmental Protection Agency Method TO-11A. According to Method TO-11A, DNPH cartridges were extracted using HPLC grade acetonitrile solvent and analyzed using HPLC techniques to detect the aldehyde derivatized DNPH complex. The detection limit for aldehyde in the outlet stream was around 0.1 μg/mL.

A 100-L Tedlar® bag fitted with luer fittings and a Teflon septa injection port served as the acetaldehyde source. The performance of the MOF was evaluated by measuring the removal of acetaldehyde. The inlet concentration of acetaldehyde was measured as 425 ppm. The MOF was exposed to the acetaldehyde for a period of 15 to 20 minutes. The acetaldehyde removal efficiency of the MOF was estimated as 99%.

### Example 6-Example 35

The procedure in Example 5 was repeated, varying the MOF or commercial material, aldehyde, and inlet concentration as shown in Tables 1 through 3. The removal efficiency of the various samples is shown for acetaldehyde, crotonaldehyde, and formaldehyde in Tables 1, 2, and 3, respectively. It is noted that in Examples 6 and 8, the outlet concentration of the aldehyde is greater than the inlet concentration. During these runs, difference between the inlet concentration and the outlet concentration is within the margin of error for the equipment used. Because of this, the catalyst efficiency is listed as 0% for Examples 6 and 8.

As shown in Tables 1 through 3, there is a significant difference in the performance of the ZnA-MOF produced using the method in Example 1 compared to the performance of the MOF produced using the method in Comparative Example 1. Though these two synthesis processes use the same organic ligand and metal ion, the Example 1 ZnA-MOF outperformed the Comparative Example 1 MOF in removal of each of acetaldehyde, crotonaldehyde, and formaldehyde. On a percentage basis, the Example 1 ZnA-MOF removed 99% of each of acetaldehyde, crotonaldehyde, and formaldehyde, compared to the Comparative Example 1 MOF removal of 0%, 5%, and 42% of acetaldehyde, crotonaldehyde, and formaldehyde, respectively. Further, per gram of MOF, the Example 1 ZnA-MOF removed 16,455 μg, 2214 μg, and 37,815 μg of acetaldehyde, crotonaldehyde, and formaldehyde, respectively. Contrasting, per gram of MOF, the Comparative Example 1 MOF removed 0 μg, 165 μg, and 2226 μg of acetaldehyde, crotonaldehyde, and formaldehyde, respectively.

TABLE 1
Removal Efficiencies for Acetaldehyde

| | Catalyst/Absorbent | Inlet | Outlet | Removal | VOC removal |
|---|---|---|---|---|---|
| Example | Type | (ppm) | (ppm) | Efficiency (%) | per gram (μg/g) |
| 5 | Example 1 | 425 | 2.8 | 99 | 16,455 |
| 6 | Comparative Example 1 | 420 | 538 | 0 | 0 |
| 7 | Comparative Example 2 | 420 | 24 | 94 | 7,535 |
| 8 | Comparative Example 3 | 420 | 471 | 0 | 0 |
| 9 | Comparative Example 4 | 420 | 393 | 7 | 324 |
| 10 | Comparative Example 5 | 420 | 417 | 1 | 54 |
| 11 | Calgon Carbon powder | 420 | 92 | 78 | 12,798 |
| 12 | Amberlyst ® 36 | 420 | 348 | 17 | 1008 |
| 13 | Zeolite Y CBV-600 | 420 | 3.7 | 99 | 12,609 |
| 14 | Zeolite Y CBV-901 | 420 | 47 | 86 | 10,593 |

TABLE 2
Removal Efficiencies for Crotonaldehyde

| | Catalyst/Absorbent | Inlet | Outlet | Removal | VOC removal |
|---|---|---|---|---|---|
| Example | Type | (ppm) | (ppm) | Efficiency (%) | per gram (μg/g) |
| 15 | Example 1 | 20.9 | 0.28 | 99 | 2206 |
| 16 | Comparative Example 1 | 20.9 | 19.8 | 5 | 165 |
| 17 | Comparative Example 2 | 20.9 | 0 | 100 | 1092 |
| 18 | Comparative Example 3 | 20.9 | 0 | 100 | 939 |
| 19 | Comparative Example 4 | 20.9 | 0 | 100 | 681 |
| 20 | Comparative Example 5 | 20.9 | 0 | 100 | 854 |
| 21 | Calgon Carbon powder | 20.9 | 0 | 100 | 2236 |
| 22 | Amberlyst ® 36 | 20.9 | 16.9 | 19 | 153 |
| 23 | Zeolite Y CBV-600 | 20.9 | 0 | 100 | 1739 |
| 24 | Zeolite Y CBV-901 | 20.9 | 0 | 100 | 1677 |

TABLE 3
Removal Efficiencies for Formaldehyde

| | Catalyst/Absorbent | Inlet | Outlet | Removal | VOC removal |
|---|---|---|---|---|---|
| Example | Type | (ppm) | (ppm) | Efficiency (%) | per gram (μg/g) |
| 25 | Example 1 | 100.9 | 1.3 | 99 | 46,402 |
| 26 | Example 1 | 859.6 | 0.31 | 100 | 37,815 |
| 27 | Comparative Example 1 | 95.2 | 54.8 | 42 | 2226 |
| 28 | Comparative Example 2 | 429.3 | 0 | 100 | 8356 |
| 29 | Comparative Example 3 | 849.3 | 0 | 100 | 17,528 |
| 30 | Comparative Example 4 | 991.4 | 4.4 | 100 | 13,070 |
| 31 | Comparative Example 5 | 991.4 | 0 | 100 | 16,469 |
| 32 | Calgon Carbon powder | 72.9 | 0 | 100 | 5011 |
| 33 | Amberlyst ® 36 | 417 | 0.26 | 100 | 5932 |
| 34 | Zeolite Y CBV-600 | 72.9 | 0 | 100 | 3897 |
| 35 | Zeolite Y CBV-901 | 72.9 | 0 | 100 | 3758 |

1. A metal-organic framework (MOF) prepared by a process comprising:
(1) mixing an organic ligand with a metal ion in a first solvent to form a first solution;
(2) adding an amine to said first solution to precipitate said MOF and form a first suspension;
(3) separating said MOF from said first suspension;
(4) drying said MOF.

2. The metal-organic framework of claim 1, wherein said organic ligand is selected from the group consisting of aminoterephthalic acid, terephthalic acid, 1,2,3-benzenetricarboxylic acid, 1,3,5-benzenetricarboxylic acid, and 2,2′-bipyridine-5,5′-dicarboxylic acid;
wherein said metal ion is selected from the group consisting of zinc, copper, cerium, nickel, manganese, platinum, and iron; and
wherein said amine is selected from the group consisting of methylamine, ethylamine, n-propylamine, iso-propylamine, n-butylamine, sec-butylamine, iso-butylamine, tert-butylamine, n-pentylamine, neo-pentylamine, n-hexylamine, pyrrolidine, cyclohexylamine, morpholine, pyridine, 8-azaphenanthrene, 1,4-diaminobenzene, and triethylamine.

3. The metal-organic framework of claim 1, wherein said adding said amine step occurs at room temperature.

4. The metal-organic framework of claim 1, wherein said separating step comprises (a) a first filtering of said MOF out of said first suspension, (b) a first washing of said MOF with a second solvent, and (c) a second filtering of said MOF.

5. The metal-organic framework of claim 4, wherein said first solvent comprises dimethylformamide, diethylformamide, or dibenzylformamide; and wherein said second solvent comprises ethanol, dimethylformamide, dichloromethane, toluene, methanol, chlorobenzene, diethylformamide, methylamine, acetonitrile, benzyl chloride, or ethylene glycol.

6. The metal-organic framework of claim 1, wherein said organic ligand comprises aminoterephthalic acid, wherein said metal ion comprises zinc, and wherein said amine comprises triethylamine.

7. The metal-organic framework of claim 1, wherein said MOF is in the form of essentially spherical particles.

8. The metal-organic framework of claim 7, wherein 90% of said particles have a diameter ranging from 10 μm to 20 μm.

9. A method of synthesizing a metal-organic framework (MOF) comprising:
(1) mixing an organic ligand with a metal ion in a first solvent to form a first solution;
(2) adding an amine to said first solution to precipitate said MOF and form a first suspension;
(3) separating said MOF from said first suspension;
(4) drying said MOF.

10. The method of claim 9, wherein said organic ligand is selected from the group consisting of aminoterephthalic acid, terephthalic acid, 1,2,3-benzenetricarboxylic acid, 1,3,5-benzenetricarboxylic acid, and 2,2′-bipyridine-5,5′-dicarboxylic acid;
wherein said metal ion is selected from the group consisting of zinc, copper, cerium, nickel, manganese, platinum, and iron; and
wherein said amine is selected from the group consisting of methylamine, ethylamine, n-propylamine, iso-propylamine, n-butylamine, sec-butylamine, iso-butylamine, tert-butylamine, n-pentylamine, neo-pentylamine, n-hexylamine, pyrrolidine, cyclohexylamine, morpholine, pyridine, 8-azaphenanthrene, and triethylamine.

11. The method of claim 9, wherein said adding said amine step occurs at room temperature.

12. The method of claim 9, wherein said separating step comprises (a) a first filtering of said MOF out of said first suspension, (b) a first washing of said MOF with a second solvent, and (c) a second filtering of said MOF.

13. The method of claim 12, wherein said first solvent comprises dimethylformamide, diethylformamide, or dibenzylformamide; and wherein said second solvent comprises ethanol, dimethylformamide, dichloromethane, toluene, methanol, chlorobenzene, diethylformamide, methylamine, acetonitrile, benzyl chloride, or ethylene glycol.

14. The method of claim 9, wherein said organic ligand comprises aminoterephthalic acid; wherein said metal ion comprises zinc; wherein said amine comprises triethylamine.

15. The method of claim 9, wherein said MOF is in the form of essentially spherical particles.

16. The method of claim 15, wherein 90% of said particles have a diameter ranging from 10 μm to 20 μm.

17. A method of removing aldehyde from a fluid stream comprising:
(1) providing a metal-organic framework (MOF) prepared by a process comprising: (a) mixing aminoterephthalic acid with a zinc nitrate solution in a first solvent to form a first solution; (b) adding triethylamine to said first solution to precipitate said MOF and form a first suspension; (c) separating said MOF from said first suspension; (d) drying said MOF;
(2) contacting said fluid stream with said MOF;
whereby greater than 90% of said aldehyde is removed from said fluid stream.

18. The method of claim 17, wherein said aldehyde is selected from the group consisting of acetaldehyde, crotonaldehyde, formaldehyde, acrolein, butyraldehyde, benzyl aldehyde, and propionaldehyde.

19. The method of claim 17, wherein said MOF is in the form of essentially spherical particles.

20. The method of claim 19, wherein 90% of said particles have a diameter ranging from 10 μm to 20 μm.

21. The method of claim 17, wherein said aldehyde comprises acetaldehyde, and wherein said acetaldehyde is removed in an amount ranging from about 13,000 μg to about 24,000 μg per gram of said MOF.

22. The method of claim 17, wherein said aldehyde comprises crotonaldehyde, and wherein said crotonaldehyde is removed in an amount ranging from about 1800 μg to about 2500 μg per gram of said MOF.

23. The method of claim 17, wherein said aldehyde comprises formaldehyde, and wherein said formaldehyde is removed in an amount ranging from about 18,000 μg to about 69,000 μg per gram of said MOF.

24. A method for embedding at least one metal-organic framework (MOF) into a cellulose acetate fiber comprising:
(i) preparing cellulose acetate fibers;
(ii) mixing the cellulose acetate fibers with a first solution comprising at least one metal ion and at least one organic ligand;
(iii) adding an amine to the first solution to form a precipitate comprising cellulose acetate fibers having at least one MOF embedded therein;
(iv) separating the cellulose acetate fibers having at least one MOF embedded therein; and optionally
(v) drying the cellulose acetate fibers having at least one MOF embedded therein.

25. The method of claim 24 further comprising the step of using a solvent to filter and wash the cellulose acetate fibers having at least one MOF embedded therein.

26. A cellulose acetate fiber having embedded therein at least one metal-organic framework (MOF).
